(19) United States
(12) Patent Application Publication
(10) Pub. No.: US 2005/0076399 A1
(43) Pub. Date: Apr. 7, 2005

(54) "Gfp-transfected clon pig, gt knock-out clon pig and methods for productions thereof

(76) Inventors: So H. Lee, Suwon-shi (KR); Woo S. Hwang, Seoul (KR); Byeong C. Lee, Gwanak-ku (KR); Sung K. Kang, Gwanak-ku (KR); Jek Y. Han, Gangnam-ku (KR); Jeong M. Lim, Seoul (KR); Chang K. Lee, Gwanak-ku (KR); Eun S. Lee, Gangwon-do (KR); Eui B. Jeung, Chungcheongbook-do (KR); Jong K. Cho, Songpa-ku (KR); Dae Y. Kim, Daegu (KR); Sang H. Hyun, Jeju-do (KR); Gab S. Lee, Gangnam-ku (KR); Hye S. Kim, Gangnam-ku (KR); Sung C. Lee, Jullabook-do (KR); Su C. Yeom, Gyeonggi-do (KR)

Correspondence Address: SHERIDAN ROSS PC, 1560 BROADWAY, SUITE 1200, DENVER, CO, 80202, US

(21) Appl. No.: 10/500,748

(22) PCT Filed: Dec. 29, 2001
(86) PCT No.: PCT/KR01/02304

(51) Int. Cl. A01K 67/027

(52) U.S. Cl. 800/17

(57) ABSTRACT

Disclosed are a cloned pig expressing green fluorescent protein (GFP) and a cloned pig having a 1,3-galactosyltransferase (GT) gene knocked out. Also, the present invention discloses methods of producing such cloned pigs, comprising the steps of establishing a somatic cell line; preparing a GFP-transfected or GT gene knock-out nuclear donor cell; producing a transgenic nuclear transfer embryo using the nuclear donor cell and a recipient oocyte; and transplanting the transgenic nuclear transfer embryo into a surrogate mother pig. The cloned pig expressing GFP of the present invention is useful for large-scale production of an animal disease model, and the GT gene knock-out cloned pig can be used as a organ donor allowing xenotransplantation in humans without hyperacute immune rejection.

### TECHNICAL FIELD

The present invention, in general, relates to a method of producing a cloned pig with a specific genetic character by gene targeting through introduction of a desired gene into somatic cells and somatic cell nuclear transfer, and pigs produced by such a method.

More particularly, the present invention relates to a cloned pig containing a specific gene, that is, green fluorescent protein (GFP) gene that encodes a protein emitting green color at a specific wavelength of light, and a method of producing such a pig. Also, the present invention is concerned with a cloned pig in which a gene responsible for the hyperacute rejection of xenografts from pigs, that is, alpha-1,3-galactosyltransferase (GT) gene, is knocked out, and a method of producing such a pig.

In addition, the present invention relates to a gene targeting method comprising effectively introducing a GFP gene or a genetically manipulated GT gene into a cell.

Further, the present invention relates to a vector capable of effectively removing a GT gene.

The present invention indicates potential large-scale production of an animal disease model through successful introduction of a heterologous GFP gene into a pig, and makes it possible to produce a GT gene knock-out pig, thereby allowing pig organs to be transplanted into a human without hyperacute xenograft rejection.

### PRIOR ART

Transgenic animal technology has been under the spotlight for the past 20 years. The transgenic techniques are overwhelmingly important in terms of being capable of producing highly valuable products, and are widely used in biomedical and biological research. The transgenic techniques can be industrially applied in a broad range of applications from production of high-quality livestock products, high value-added pharmaceutically-active substances, animals having improved resistance to various pathogens and animal disease models, to genetic therapy.

Owing to its properties of facilitating labeling of chromosomal proteins and tagging of a specific region of chromosomal DNA, being capable of associating with many cytoplasmic proteins and being non-toxic, green fluorescent protein (GFP) gene, which is typically used at a gene targeting step to produce a transgenic animal, is widely used for expressing cognate cytoskeletal filaments in living cells. In 1994, Chalfie et al. observed various molecular biological changes in living cells including porcine embryos using GFP obtained from Aequorea victoria as a fluorescent indicator. Since then, enhanced GFP (EGFP) has been developed and utilized as a marker in several transgenic animals.

As a technique of introducing a heterologous gene into a cell to produce transgenic animals, pronuclear microinjection, which was suggested by Gordon et al., is characterized by direct injection of a heterologous gene into a pronucleus of a fertilized oocyte, and widely applied to experimental animals including mice. However, there are significant disadvantages with the pronuclear microinjection method, as follows. When pronuclear microinjection is applied to industrial animals, production yield of transgenic animals is very low (0.5% in bovine, 1.5% in pigs, and 2.5% in sheep). In addition, genetic mosaicism occurs in most cases. To overcome these problems, an alternative animal cloning technique was suggested, which employs somatic cells transfected with a heterologous gene. The transgenic animal cloning technique can effectively produce transgenic cloned animals by generating reconstructed fertilized embryos with 100% transfection efficiency and without genetic mosaicism through nuclear transfer of only somatic cells transfected with a heterologous gene, and then transplanting the reconstructed embryos into surrogate mothers. In addition, sex of the transgenic animals can be artificially determined by analyzing in advance sex chromosomes of the transfected somatic cells, thereby maximizing their industrial usefulness.

When intended to produce transgenic pigs by somatic cell nuclear transfer, preferentially, a desired gene should be isolated and a vector carrying the desired gene should be constructed, and a molecular biological technique for introduction of the desired gene into somatic cells should be used along with a somatic cell cloning technique. The gene is typically isolated from a pig genomic DNA library by screening. The vector may be prepared according to intended use with consideration of an exogenous promoter, size of a gene of interest, positive or negative selectable markers, etc. The gene is introduced into nuclear donor cells by transfection using a biochemical method, a physical method, or virus-mediated gene transfer. Examples of the biochemical method include calcium precipitation using calcium ions as a vehicle, lipofection using a cationic lipid that is a plasma membrane component, and a method using a non-lipid cationic polymer. Such transfection methods have been widely used owing to their simplicity, effectiveness and stability. The physical method includes electroporation, gene gun and intracytoplasmic microinjection. The virus-mediated gene transfer can be achieved by cloning a desired DNA into viral genome of adenovirus or retrovirus and then infecting cells with the resulting virus. The somatic cell cloning technique is disclosed in International Pat. Application No. PCT/KR00/00707 filed on Jun. 30, 2000 by the present applicant, entitled “Method for Producing Cloned Cows”, where somatic cell cloning is achieved by removing a nucleus containing genetic material from a cow oocyte and then injecting a nucleus from a different cell into the enucleated unfertilized oocyte. The resulting fertilized embryo is called “reconstructed embryo”. After being post-activated and cultured in vitro, the reconstructed embryo is transferred into a surrogate mother to produce live offspring.

Organ transplantation in humans is a useful tool for treating organ-related incurable diseases, and has gradually increased for the past over 10 years. Relative to such increase of organ transplantation procedures, however, for the same period, the number of patients wanting to receive organ transplantation has increased three times. This is due to an unbalance of supply and demand, meaning shortage of human organs for surgical transplantation. Although organ supply sources are seriously deficient, there is still no satisfactory method capable of solving the problem. Efforts to overcome such lack of organs for surgical transplantation in humans have been tried, which include development of artificial organs by medical engineering approaches and production of transgenic animals. In case of obtaining organs capable of substituting for diseased human organs from transgenic animals, pigs are typically selected as organ donors because of having similarity to humans in terms of physiological properties, size of the blood vessel system, and even diameter of erythrocytes. Moreover, the use of pig organs is not problematic ethically, in comparison with primates.

However, when pig organs are transplanted into humans, transplantation is not generally successful owing to hyperacute immune rejection against the xenografts, thus causing severe side effects in recipient patients. Binding of an anti-Gal antibody in human blood to the xenoantigen gal epitope on cells or tissues of pigs induce the hyperacute immune rejection. Several methods for overcoming such immune rejection response have been suggested, including genetic manipulation to suppress the activity of complement proteins in humans, and continuous administration of a drug capable of lowering the activity of the human immune system. However, such methods were proved to be unsafe because severe impairment of the immune system made patients vulnerable to infection by pathogenic microorganisms or viruses. In contrast, in the present invention, alpha-1,3-galactosyltransferase (GT) gene, responsible for the formation of the xenoantigen, is disrupted in advance by gene targeting, thereby making it possible for xenografts from the resulting transgenic pig to be successfully transplanted into humans without hyperacute immune rejection of the xenografts, as well as not impairing the protective immune response in humans.

### DISCLOSURE OF THE INVENTION

Based on the conventional techniques, the present invention provides methods of producing a cloned pig expressing green fluorescent protein (GFP) and an alpha-1,3-galactosyltransferase (GT) gene knock-out cloned pig, and pigs produced by such methods, by gene targeting using a transfection method and somatic cell nuclear transfer.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a photograph of a somatic cell expressing GFP;

FIG. 2 is a photograph of a nuclear transfer (NT) embryo obtained by transferring a somatic cell expressing GFP into a recipient oocyte;

FIG. 3 is a photograph of a GFP-expressing nuclear transfer (NT) embryo at the blastocyst stage;

FIG. 4 is a photograph showing transplantation of a transgenic nuclear transfer embryo into the oviduct of a surrogate mother;

FIG. 5 is a photograph showing a screening result of the primary pig genomic BAC library pool for GT gene;

FIG. 6 is a photograph showing a result of screening the secondary pig genomic BAC library pool for GT gene;

FIG. 7 is a photograph showing a result of screening the tertiary pig genomic BAC library pool for GT gene;

FIG. 8 is a photograph showing a result of restriction mapping of a cloned GT gene; and

FIG. 9 is a schematic view of a vector for targeting of GT gene.

### BEST MODES FOR CARRYING OUT THE INVENTION

The present invention is characterized by providing a transgenic cloned pig expressing a desired gene or having another desired gene knocked out, where the cloned pig is produced by gene targeting using a transfection method and somatic cell nuclear transfer.

In detail, the present invention provides a transgenic cloned pig expressing GFP or having a GT gene knocked out by generating GPF-expressing or GT gene knock-out somatic cells using a transfection method, yielding reconstructed embryos by nuclear transfer, and transferring the reconstructed embryos into a surrogate mother.

First, a method of producing a cloned pig expressing a GFP gene comprises the steps of (a) preparing a nuclear donor cell by culturing a cell line collected from a pig; (b) mixing a DNA construct carrying a GFP gene and a lipid component or non-lipid cationic polymer vehicle to form lipid (or cationic polymer)-DNA complexes, and adding the resulting complexes to a culture medium of the nuclear donor cell and further culturing the nuclear donor cell to introduce the GFP gene and express the GFP gene therein; (c) transferring the transfected nuclear donor cell into an enucleated pig recipient oocyte to generate a transgenic nuclear transfer (NT) embryo, and activating the NT embryo; and (d) transplanting the NT embryo into a surrogate mother pig to produce live offspring.

The method of producing a cloned pig expressing GFP is described in more detail with respect to each step, as follows.

Step 1: Preparation, In Vitro Culturing and Maintenance of Nuclear Donor Cells

To produce transgenic animals expressing GFP using a somatic cell nuclear transfer technique, nuclear donor cells are needed. Several kinds of cells, including somatic cell-derived cells and fertilized embryo-derived cells, are used as nuclear donor cells supplying nuclei in a nuclear transfer procedure. Of them, somatic fibroblasts isolated from pig fetuses are typically used. The fibroblasts have advantages in that a plurality of cells can be obtained at the initial step of fibroblast cell isolation, and they are relatively easy to culture and manipulate in vitro.

To isolate fetal fibroblasts mainly used as nuclear donors, crown-rump length of fetuses obtained from pregnant sows is measured, and length of gestation of the sows is calculated with reference to its breeding history. The fetal pigs are isolated by removing the fetal membrane, and then cutting the umbilical cord near the fetuses. Then, the fetal pigs are washed several times with phosphate-buffered saline (PBS) containing antibiotics and bovine serum albumin (BSA). After surgically removing the four legs, head and viscera from the body of the fetus, the body is again washed with PBS. To obtain fibroblasts from tissues, remaining tissues are mechanically finely ground, and explant cultures are prepared, or trypsin-EDTA is added to the ground tissues to release cells.

Thereafter, to prepare nuclear donor cells, the isolated fetal fibroblasts are incubated at 38° C. under 95% humidity and 5% CO₂. When the culture is 90-100% confluent, the cells are subcultured, and the surplus cells are cryo-preserved.

Step 2: Gene Targeting by Introduction of GFP Gene into Somatic Cells

pEGFP-N1 vector (Clontech Laboratories Inc., Palo Alto, Calif.), which is commercially available, is used in targeting of GFP gene. The pEGFP-N1 vector expresses a modified form of wild-type GFP, where the modified GFP has a high expression level and emits bright fluorescence. The vector is introduced into somatic cells using a biochemical vehicle, such as FuGENE 6 (Roche Diagnosis Corp. IN, USA), LipofectAmine Plus (Life Technologies) or ExGen 500 (MBI Fermentas). The FuGENE 6 transfection reagent, which is a multi-component lipid based reagent, is advantageous in terms of having high transfection efficiency in a variety of cell types and low cytotoxicity, functioning both in the presence or absence of serum, and being easy to optimize its complex formation with DNA at a minimum volume. LipofectAmine Plus, which is a cationic lipid, and ExGen 500, which is a non-lipid cationic polymer, was reported to have high transfection efficiency in a variety of cell types.

Cells into which a GFP gene are to be introduced are grown under optimal conditions, and subcultured by treatment with trypsin-EDTA to dissociate attached cells into single cells. One day before transfection, the subcultured cells are fed with a fresh culture medium, and the medium is again exchanged with a fresh medium 4 hrs before transfection. When the culture reaches an optimal cell density according to the biochemical vehicles, the GFP gene is introduced into the cultured cells.

In the present invention, lipid and non-lipid biochemical vehicles are used for targeting of the GFP gene by introduction of the GFP gene into nuclear donor cells. The GFP gene was mixed with a lipid or non-lipid vehicle to form complexes, and the resulting complexes were introduced into nuclear donor cells. To effectively introduce the GFP gene into the cells, several parameters including amount of GFP gene DNA, volume of the vehicle, cell density, transfection time and addition or no addition of serum are selected, and optimized, thereby maximizing introduction efficiency and expression level of the GFP gene.

Step 3: Selection, Proliferation and Cryo-Preservation of Nuclear Donor Cells Transfected with GFP Gene

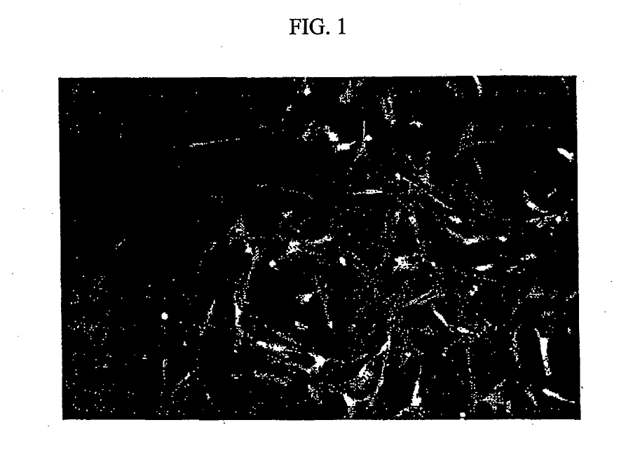

After being transfected with the GFP gene, nuclear donor cells are cultured for 3-5 days until the culture is completely confluent, where the GFP gene is integrated into chromosomes of the cells. Then, the cells are trypsinized, and the resulting single cells are observed under a fluorescence microscope equipped with a UV filter to select only green colored cells. In addition, nuclear donor cells transfected with the GFP gene are selected by in vitro culturing in the presence of a specific antibiotic. The pEGFP-N1 vector, carrying a GFP gene, contains a neomycin-resistant gene that is used as a positive selectable marker. The neomycin-resistant gene is introduced into the cells along with the GFP gene, and expresses a neomycin-resistant protein in the cells. Therefore, when the targeted cells are cultured in a culture medium containing neomycin, only cells transfected with the vector survive, and cells not transfected with the vector die due to action of neomycin, resulting in proliferation of only the transfected cells in culture dishes (FIG. 1).

Such selection using antibiotics may be effectively achieved by determining an optimal treatment concentration of antibiotics. The targeted cells are selected through treatment with neomycin for 2-3 weeks, where neomycin is added to the culture medium at a concentration of 200-800 μg/ml at intervals of 4-5 days. Cell proliferation pattern varies according to cell types. However, because cells are generally proliferated from one cell, the targeted cells should be proliferated at least up to a level required at the next step.

After the selection of the targeted cells is finished, the selected cells are cultured in a normal culture medium, where suitable growth factors and apoptosis-suppressing agents are added to the medium to induce rapid proliferation and reduce unnecessary loss of cells by apoptosis. For effective preservation of the proliferated cells, an optimal condition for cell storage is established, and the proliferated cells are cryo-stored at each passage.

Step 4: Production of a Reconstructed Embryo by Somatic Cell Nuclear Transfer

To produce a transgenic animal having the genetic character of the transfected nuclear donor cells, the present invention employs a cloning technique by somatic cell nuclear transfer, thereby generating a reconstructed embryo. Primarily, recipient oocytes are prepared by in vitro maturation of immature oocytes, as follows. Pig ovary is collected mainly in a slaughterhouse, tested for abnormalities, and washed three times with a proper washing solution. Then, immature oocytes are matured in vitro by culturing in a culture medium for maturation of the immature oocytes, that is, bovine serum albumin-free NCSU23 medium (North Carolina State University 23 (NCSU23-M), see Table 1), containing 10% porcine follicular fluid (PFF), gonadotropic hormones (GTH), pregnant mare serum gonadotropin (PMSG) (Intervet Folligon), human chorionic gonadotropin (hCG) (Intervet Chorulon), and epidermal growth factor (GF) of 10 ng/ml.

For nuclear transfer, recipient oocytes, nuclear donor cells, and pipettetes for cutting, enucleation and injection are prepared. Culture media are prepared using NCSU23 (NSCU23-W, see Table 2) washing medium as a basal medium. Each of recipient oocytes is put into NCSU23-W medium supplemented with 0.1% hyaluronidase to remove cumulus cells surrounding oocytes. The completely denuded oocytes are washed with a microdrop of NCSU23-W medium.

The denuded oocytes are fixed with a holding pipette, a portion of the zona pellucida at an upper part of the first polar body is cut using a sharp pipette to give a slit.

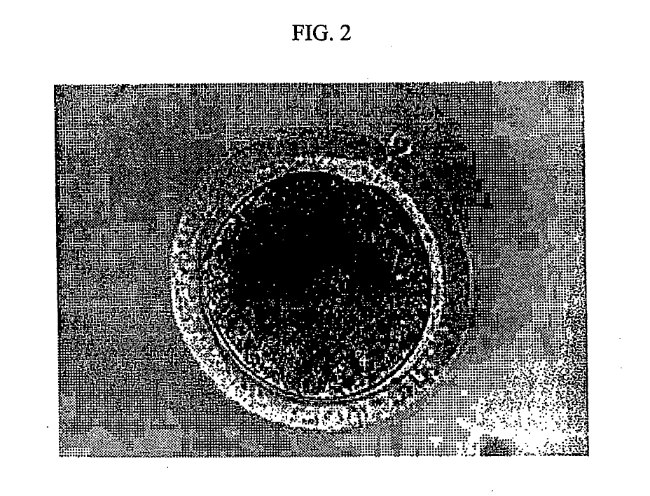

Using the pipette used in the cutting of the zona pellucida, a portion of cytoplasm including the first polar body is removed by squeezing through the slit to generate enucleated oocytes. The enucleated oocytes are washed with NCSU23-W medium, and placed in a microdrop of NCSU23-M medium until nuclear transfer. The prepared nuclear donor cells are transferred to the enucleated recipient oocytes by aspirating the donor cells using an injection pipette after positioning the slit made on the zona pellucida of the oocytes to a straight line to the holding pipette, and injecting each of the donor cells into the perivitelline space of each of the enucleated oocytes through the slit, resulting in production of nuclear transfer embryos (FIG. 2).

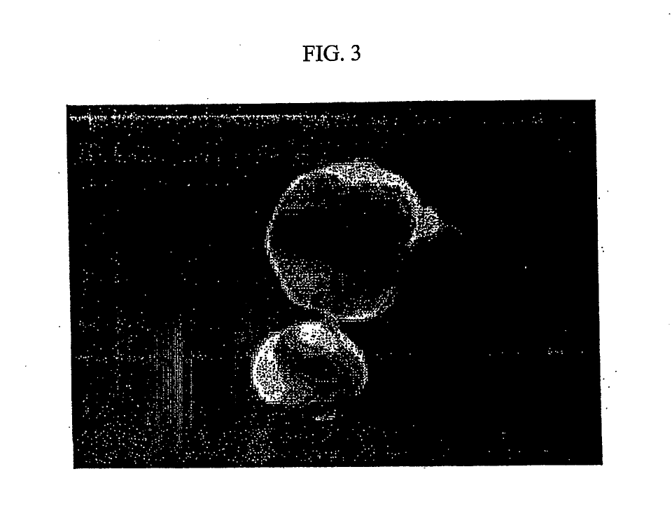

The nuclear transfer embryos are subjected to electrofusion, in which the enucleated oocytes are electrically fused with the donor cells with a single DC pulse of 1.8 kV/cm for 30 μsec using a BTX Electro Cell Manipulator (ECM001, BTX, USA). The electrofused reconstructed embryos are washed with NCSU23-W medium, and incubated in NCSU23 culture medium (NCSU23-D, see Table 3). On day 4 after incubation, the NCSU23 medium is supplemented with 10% serum. On day 7, the reconstructed embryos are evaluated for development to the blastocyst stage and GFP expression (FIG. 3).

TABLE 1
Composition of NCSU-M

| Components | Conc. | |
|---|---|---|
| NaCl | 108.73 | mM |
| KCl | 4.78 | mM |
| HEPES | 10 | mM |
| CaCl₂ | 1.70 | mM |
| KH₂PO₄ | 1.19 | mM |
| MgSO₄ | 1.19 | mM |
| NaHCO₃ | 25.07 | mM |
| Glucose | 5.55 | mM |
| Glutamine | 1.00 | mM |
| FCS | 10% | (v/v) |

TABLE 2
Composition of NCSU-W

| Components | Conc. | |
|---|---|---|
| NaCl | 108.73 | mM |
| KCl | 4.78 | mM |
| HEPES | 10 | mM |
| CaCl₂ | 1.70 | mM |
| KH₂PO₄ | 1.19 | mM |
| MgSO₄ | 1.19 | mM |
| NaHCO3 | 25.07 | mM |
| Glucose | 5.55 | mM |
| Taurine | 7.00 | mM |
| Hypotaurine | 5.00 | mM |
| Glutamine | 1.00 | mM |
| FCS | 10% | (v/v) |

TABLE 3
Composition of NCSU-D

| Components | Conc. | |
|---|---|---|
| NaCl | 108.73 | mM |
| KCl | 4.78 | mM |
| CaCl₂ | 1.70 | mM |
| KH₂PO₄ | 1.19 | mM |
| MgSO₄ | 1.19 | mM |
| NaHCO₃ | 25.07 | mM |
| Glucose | 5.55 | mM |
| Taurine | 7.00 | mM |
| Hypotaurine | 5.00 | mM |
| Glutamine | 1.00 | mM |
| FCS | 10% | (v/v) |

Step 5: Transplantation of Reconstructed Embryos to Surrogate Mother Pigs and Production of Live Offspring

Surrogate mother pigs suitable for transplantation of the reconstructed embryos and capable of developing the reconstructed embryos to normal fetuses are selected. The best time for the transplantation is determined by monitoring the estrus cycles of the selected sows. Generally, it is suitable for fertilization to be performed about 30-40 hours after a sow shows behavioral signs of estrus. Therefore, based on a suitable fertilization period, a proper time for embryo transplantation is calculated with consideration of time required for in vitro development of the reconstructed embryos.

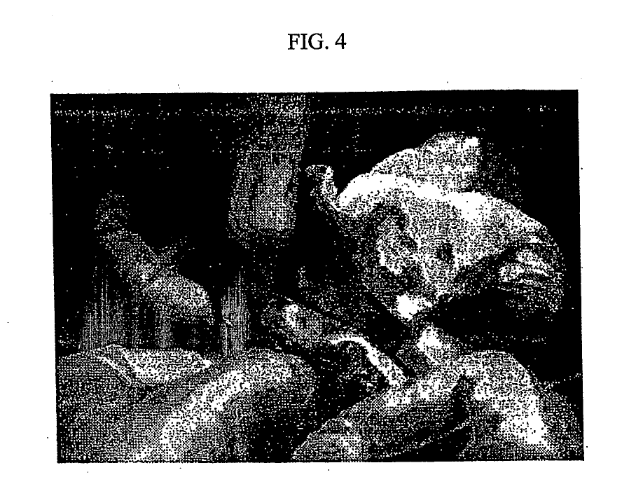

The reconstructed embryos are transferred to a surrogate mother pig by injecting the reconstructed embryos 2 cm deep in the oviduct, close to the ovary, after opening the abdomen of the surrogate mother by laparectomy (FIG. 4). 4 weeks after embryo transplantation, the sow is evaluated for pregnancy by ultrasound. After that, the ultrasonic diagnosis is carried out every two weeks to monitor the pregnancy of the surrogate mother and growth state of fetuses.

If piglets are not delivered even though the calving process exceeds 30 min, an experienced assistant should help calving of a mother sow. When the expected calving date is passed, calving is induced by injecting a hormone preparation into the mother sow, or by surgical operation such as Caesarean section.

Based on the method described above, using fetal pig fibroblasts as nuclear donor cells, the present inventors produced a reconstructed embryo expressing GFP by nuclear transfer of somatic fibroblast cells transfected with a GFP gene to enucleated recipient embryos, and in vitro culturing of the resulting nuclear transfer embryos for 7 days to allow their development to the blastocyst stage. The reconstructed embryo was designated “SNU-P1 [Porcine NT Embryo]”, and deposited at an international depositary authority, KCTC (Korean Collection for Type Cultures; KRIBB, 52, Oun-dong, Yusong-ku, Taejon, Korea) on Dec. 27, 2001, under accession number KCTC 10145BP. The present inventors obtained normal cloned offspring by transferring the reconstructed embryo to surrogate mother pigs.

On the other hand, a method of producing a GT gene-knockout cloned pig comprises the steps of (a) preparing a nuclear donor cell by culturing a somatic cell line collected from a pig; (b) isolating a GT gene clone from a pig genomic BAC library, and constructing a gene targeting vector using the isolated GT gene, wherein the vector carries a GT gene modified by substituting a portion of a wild-type GT gene with a gene encoding a selectable marker by homologous recombination to suppress expression of a normal GT protein; (c) mixing the vector with a lipid or non-lipid component to form lipid (or non-lipid)-DNA complexes, and adding the resulting complexes to a culture medium of the nuclear donor cell to allow gene targeting by introducing the recombinant GT gene into the nuclear donor cell; (d) transferring the nuclear donor cells transfected with the recombinant GT gene into an enucleated pig recipient oocyte to generate a transgenic nuclear transfer (NT) embryo, and activating the NT embryo; and (e) transplanting the NT embryo into a surrogate mother pig to produce live offspring.

The method of producing a cloned pig expressing a GFP gene is described in more detail with respect to each step, as follows.

Step 1: Preparation, In Vitro Culturing and Maintenance of Nuclear Donor Cells

To produce transgenic animals having a GT gene knocked out by somatic cell nuclear transfer, nuclear donor cells are needed. Nuclear donor cells are prepared according to the same method as in Step 1 of the method of producing a cloned pig expressing a GFP gene.

Step 2: Isolation of GT Gene

A GT gene is isolated by screening a pig genomic BAC library comprising three pools in total (Human Genome Mapping Project Inc., Great Britain). Primers to be used for the screening are prepared using the known pig GT cDNA sequence (GeneBank Accession No.: AF221517). To test specificity of primers and PCR method using the primers, PCR is carried out using pig genomic DNA and the primers, giving a positive PCR result. Using the primers, the three pig genomic BAC library pools are screened by PCR, and a single clone is obtained by PCR in which an amplified DNA fragment has an expected size. Then, the obtained GT gene clone is verified by Southern blotting.

Step 3: Construction of a Gene Targeting Vector Carrying a Knocked Out GT Gene and Introduction of the Vector into Nuclear Donor Cells

A gene targeting vector is prepared using the obtained GT gene clone. A GT gene is disrupted by substituting a portion of a GT gene with a gene encoding a selectable marker through homologous recombination, thereby preventing production of a normal GT protein.

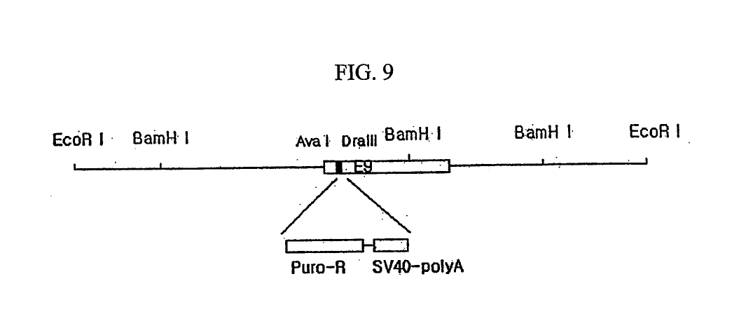

To effectively select targeted cells, the vector is constructed not to have exogenous promoters by a promoter trap method. The vector comprises a nucleic acid sequence corresponding to a part of intron 8, exon 9 and a part of intron 9 of a GT gene, and a nucleic acid sequence encoding a puromycin-resistant gene linked to a SV40 poly(A) sequence, wherein the puromycin-resistant gene substitutes a nucleic acid sequence corresponding to an AvaI-DraIII fragment of the exon 9. The puromycin-resistant gene linked to SV40 poly(A) is inserted to the exon 9 of the GT gene by homologous recombination, thereby disrupting the GT gene (FIG. 9). The gene targeting vector is introduced into nuclear donor cells using FuGENE 6 mentioned in the method of producing a cloned pig expressing GFP.

The resulting nuclear donor cells are cultured in a culture medium containing puromycin for 1-2 weeks to select targeted somatic fibroblasts. Thereafter, the selected somatic fibroblasts are confirmed by a method common in the art including Southern blotting and PCR.

Step 4: Production of a Reconstructed Embryo by Somatic Cell Nuclear Transfer

This step is carried out according to the same procedure in Step 4 of the method of producing a cloned pig expressing GFP.

Step 5: Transplantation of the Reconstructed Embryos to Surrogate Mother Pigs and Production of Live Offspring

This step is carried out according to the same procedure in Step 5 of the method of producing a cloned pig expressing GFP.

Based on the method described above, using pig fetal fibroblasts as nuclear donors, the present inventors produced a reconstructed embryo having a knocked out GT gene, by nuclear transfer of somatic fibroblast cells transfected with a vector having a knocked out GT gene into enucleated recipient embryos. The reconstructed embryo is designated “SNU-P2 [Porcine NT Embryo]”, and deposited at an international depositary authority, KCTC (Korean Collection for Type Cultures; KRIBB, 52, Oun-dong, Yusong-ku, Taejon, Korea) on Dec. 27, 2001, under accession number KCTC 10146BP. The present inventors obtained normal cloned offspring by transferring the reconstructed embryo “SNU-P2” to surrogate mother pigs.

The present invention will be explained in more detail with reference to the following example in conjunction with the accompanying drawings. However, it will be apparent to one skilled in the art that the following example is provided only to illustrate the present invention, and the present invention is not limited to the example.

### EXAMPLE 1

### Preparation, In Vitro Culturing and Maintenance of Nuclear Donor Cells

After collecting pregnant pig uteruses, the following operations were performed under an aseptic environment. 30 day-old fetuses having a crown-rump length of about 25 mm were mainly isolated. The fetuses surrounded by the amniotic membrane were isolated aseptically. After being removed of heads, four legs and viscera, the fetuses were washed several times with a phosphate-buffered solution containing some kinds of antibiotics and antimycotics. Fetal tissues were isolated from the fetuses in dishes containing 0.25% trypsin-EDTA using surgical scissors. The isolated fetal tissues were incubated in a 5% CO₂ incubator at 38° C. for 30 min. Thereafter, trypsin was eliminated from the fetal pig tissues by several centrifugations, and the fetal pig tissue explants were then cultured in 10% FCS-containing DMEM (Dulbecco's Modified Eagle's Medium).

When reaching 90-100% confluency, cells were subcultured, and the surplus was cryo-preserved. The cryo-preserved cells were used as nuclear donors in somatic cell nuclear transfer, and subculture was carried out in a culture medium containing growth factors and an apoptosis suppressor to stimulate growth of cells and suppress cell death.

### EXAMPLE 2

### Screening of Pig BAC Genomic Library for GT Gene

Before screening a pig BAC genomic library, to obtain a positive control, pig genomic DNA was primarily prepared as follows. After obtaining about 5 g of ovary from a 6 month-pregnant Landrace sow, the obtained ovary was finely cut and ground in a mortar containing liquid nitrogen to destroy tissues. The ground tissue was treated with proteinase K at a concentration of 11 mg/ml and subjected to phenol extraction, thus giving pig genomic DNA.

Screening of pig GT gene was carried out using a pig BAC genomic library. To obtain single clones, the library comprising three pools were screened sequentially. The primary pool is composed of 17 vials alphabetically marked from A to R (excluding K), the secondary pool is composed of 96-well plates with each of 15 individual pools, and the tertiary pool consists of 384-well plates for each pool of the secondary pool. First, using the known pig GT cDNA (GeneBank Accession No.: AF221517), a PCR primer set consisting of a sense primer and an antisense primer was prepared: pig GT5 (5′-GAT CAA GTC CGA GAA GAG GTG GCA A-3′); and pig GT3 (5′-TCC TGG AGG ATT CCC TTG AAG CAC T-3′). When performing PCR using pig genomic DNA with the primer set, the expected PCR product is 342 bp in size. To obtain a positive control to identify GT signals in screening, PCR was carried out using the following PCR mixture and under the following conditions. A PCR mixture was composed of 1 unit of Taq DNA polymerase, 10 mM dNTPs, 200 mM Tris-Cl (pH 8.8), 100 mM KCl, 100 mM (NH₄)₂SO₄, 1% TritonX-100, 1 mg/ml of BSA, 100 ng/μl of the pig genomic DNA and 2 μl of the primer set (40 pmol/μl of a sense primer and 40 pmol/μl of an antisense primer) in a total volume of 20 μl. PCR conditions included denaturation at 95° C. for 5 min, and 40 cycles of denaturation at 95° C. for 1 min, annealing at 55° C. for 1 min and extension at 72° C. for 1 min 30 sec, followed by final extension at 72° C. for 15 min. The resulting PCR reaction mixture was analyzed by electrophoresis on an agarose gel.

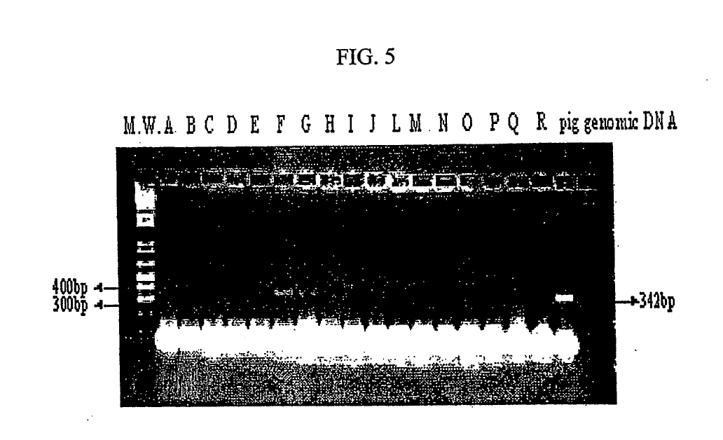

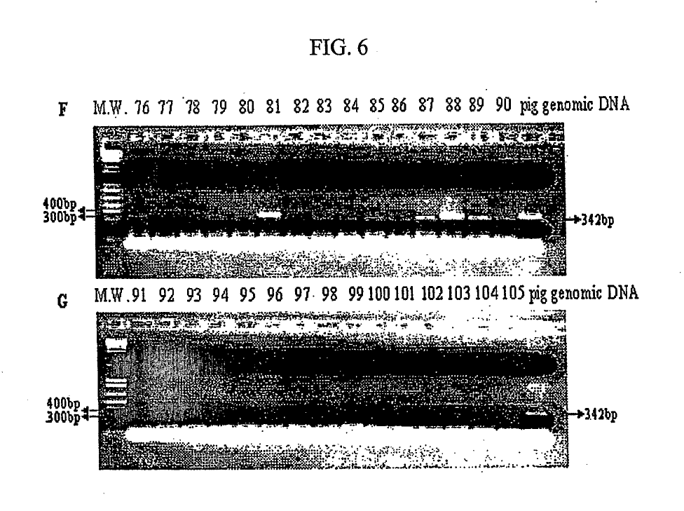

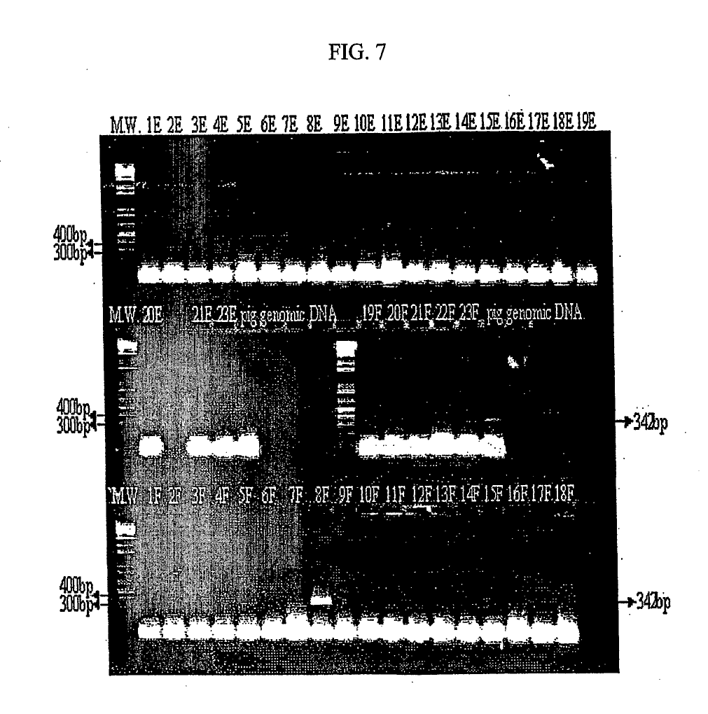

As a result of the PCR using the pig genomic DNA (100 ng/μl), a PCR product was identified to be 342 bp in size, and was used as a positive control in screening the pig BAC genomic library. Using the primary pool (17 pools of A to R, no K) of the pig BAC genomic library, PCR was carried out with the primer set of pig GT5 and pig GT3 under the same condition as the PCR using the pig genomic DNA. A PCR product having the identical size to that from the PCR using the pig genomic DNA, that is, 342 bp, was obtained in pools F and G (FIG. 5). The secondary pool (F: 76 to 90 plates; and G: 91 to 105 plates, each consisting of 15 pools) corresponding to the F and G pools showing a positive signal in the primary pool were screened by PCR under the same condition as described above, resulting in production of an amplified product having the identical size to that of the PCR using the pig genomic DNA. In this screening of the secondary pool, a PCR product of 342 bp in size was found in 81 and 82 of the F pool, and 91 of the G pool (FIG. 6). Among the selected pools, the 88 pool showed the strongest signal. When performing PCR using the tertiary pool corresponding to the 88 pool, consisting of a 384-well plate (1A to 24P) in which each well contains a single clone, the same signal as in PCR using the pig genomic DNA was found in 8F (FIG. 7).

### EXAMPLE 3

### Construction of a Vector Carrying a Knocked Out GT Gene

A rough restriction map of pig GT gene (GeneBank Accession No.: AF221517, 3.9 kb) was obtained using the Webcutter program (http://www.firstmarket.com/firstmarket/cutter/). A probe for southern hybridization, below, was prepared as follows. A DNA fragment of 351 bp in size, which corresponds to a part of the pig GT gene, was obtained by PCR and purified by gel electro-elution after electrophoresis on a 8% PAGE gel, and then labeled with α-³²P[dCTP] using a random primer labeling kit (Life Technologies, USA).

To isolate BAC DNA containing pig GT gene, 1 μl of cloned E. coli from the 8F of the tertiary pool identified in Example 2 was primarily inoculated in 3 ml LB broth (CM+), and incubated at 37° C. with agitation of 300 rpm for 12 hrs. Then, the cultured E. coli was inoculated again in 500 ml LB broth (CM+), and incubated for 16 hrs under the same condition. BAC DNA from the large-scale cultured E. coli was purified using a large-construct kit (Qiagen, Germany). Thereafter, 5 μg of the obtained BAC DNA was digested with 10 units of EcoRI, HindIII, BamHI and NotI for 3 hrs, and electrophoresed on a 1% agarose gel at 50V for 12 hrs. The resulting gel was immersed in a denaturating solution (0.5 M NaOH, 1.5 M NaCl) for 15 min and then in a neutralization solution (0.5 M Tris-Cl, 1.5 M NaCl, pH 8.0) for 15 min, and separated DNA fragments on the gel were transferred to a nylon membrane using a vacuum transfer. After being prehybridized for 3 hrs, the nylon membrane was hybridized with the prepared probe for 16 hrs. Then, the membrane was exposed to an X-ray film to identify a BAC DNA fragment containing a pig GT gene.

The identified BAC DNA fragment was cloned to pUC 19, as follows. pUC 19 vector was digested with EcoRI for 1 hr 30 min, purified by phenol/chloroform extraction, and stored at −20° C. until use. The BAC DNA fragment was mixed with 100 ng of the pUC 19 vector digested with EcoRI, 10× ligation buffer and 2 μl of T4 DNA ligase (10 units/Ξl) in a microtube, followed by incubation of 16 hrs at 15-16° C. to perform ligation.

200 μl of competent cells was added to 10 μl of the ligation mixture, and the mixture was placed on ice for 30 min, heat-shocked at 42° C. for 90 sec, and supplemented with 800 μl of LB broth, followed by incubation of 45 min at 37° C. Thereafter, the cells were plated on LB plates containing ampicillin as well as IPTG and X-gal and incubated at 37° C. overnight. White colonies were selected and incubated, and evaluated for harboring a desired DNA fragment by PCR.

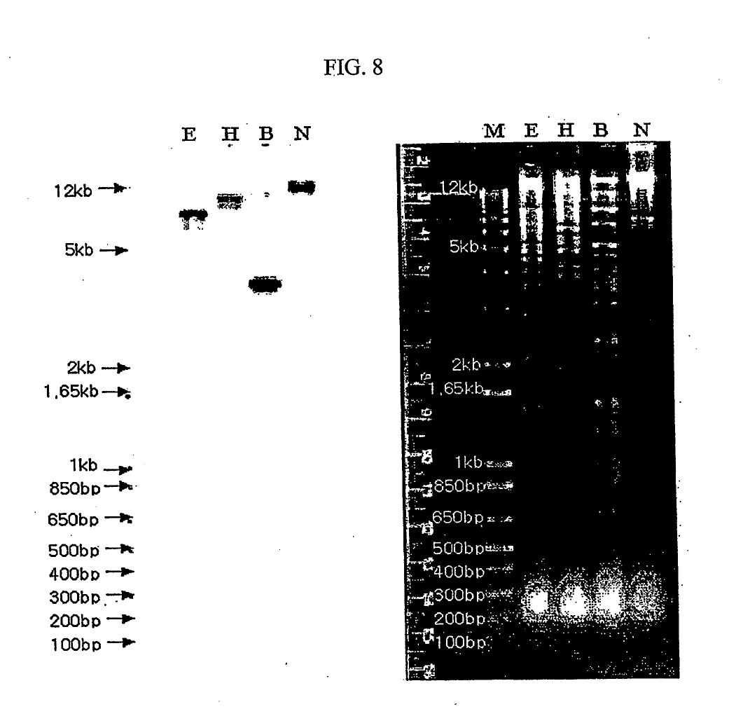

As a result of restriction mapping of the cloned pig GT gene, exon 9 was found not to have three restriction enzyme recognition sites for EcoRI, HindIII and NotI, having only a BamHI site. The cloned BAC DNA fragment containing pig GT gene was treated with each of EcoRI, HindIII, BamHI and NotI, and separated on a 1% agarose gel, where DNA bands of various sizes were found (FIG. 8). The gel was subjected to Southern hybridization. As a result, the DNA fragments containing exon 9 of the pig GT gene except for the BamHI fragment were found to be present as a single band, and have a molecular weight of about 8 to 12 kb. Particularly, the EcoRI fragment was about 8 kb in size, and contained exon 9 of the pig GT gene and a part of two introns adjacent to exon 9.

Therefore, after cleaving the cloned pig BAC DNA with EcoRI, the resulting EcoRI fragment was subcloned. Thereafter, a vector for gene targeting was prepared using the subcloned EcoRI fragment, as follows. To increase selection efficiency of targeted cells, the vector for gene targeting was prepared using a promoter trap strategy. The subcloned pig GT gene (1 μg) and a plasmid containing a puro cassette (Clontech) were digested with AvaI and DraIII, and HindIII and BamHI, respectively, at 37° C. for over 2 hrs. The digested products were treated with Klenow fragment DNA polymerase and dNTP to form blunt ends, followed by purification using a DNA elution kit (Qiagen, Germany) after electrophoresis on a 1% agarose gel. The purified GT gene fragment was ligated to a puromycin-resistant gene-SV40 poly(A) fragment using T4 DNA ligase, thereby giving a gene targeting vector (FIG. 9).

### EXAMPLE 4

### Gene Targeting by Introduction of GFP Gene and Disrupted GT Gene into Fetal Fibroblasts

Pig fetal fibroblasts for gene targeting were prepared as follows. When grown to complete confluency in 60-mm culture dishes, fetal fibroblasts were washed with phosphate-buffered saline once after eliminating the culture medium, treated with 0.25% trypsin-EDTA, resuspended in 2 ml of a culture medium containing 10% FCS, and plated in 35 mm culture dishes. Next day, when the culture was reached 50-90% confluency, transfection of the fibroblast cells with GFP gene was performed.

When using FuGENE 6, 1 μg of a DNA sample and 3 μl of FuGENE 6 were introduced into each well of a 35-mm culture dish, containing fibroblasts. First, 97 μl of a serum-free culture medium was aliquotted into Eppendorf tubes. 1 μg of pEGFP-N1 vector DNA and 3 μl of FuGENE 6 were sequentially added to each tube, followed by pulse centrifugation for 10 sec at 3000 rpm. After being incubated at room temperature for 15 min, 100 μl of the mixture was added to each well of the 35 mm culture dish, and the dish was swirled and incubated in a CO₂ incubator. The cationic liposome LipofectAmin plus (Life Technologies) has an advantage in terms of having high transfection efficiency even when using a small amount of DNA. Phosphate-buffered saline and an FCS/antibiotics-free culture medium were pre-warmed at 37° C. 30 min before use. After adding pEGFP-N1 vector DNA to an Ependorf tube in a clean bench, 100 μl of a serum-free culture medium or Opti-MEM and 4 μl of Plus reagent were mixed, and added to the tube. After well mixing using a pipette, the mixture was incubated at room temperature for 15 min. During the incubation of the DNA mixture, a 6-well plate containing 90%-confluent fetal fibroblasts was washed twice with the phosphate-buffered saline. After adding 0.8 ml of the serum-free culture medium to each well, the DNA mixture was added to each well, and the plate was swirled, followed by incubation in a CO₂ incubator. Separately, when transfection was carried out using the cationic polymer reagent ExGen 500 (MBI Fermentas), 2 μl of pEGFP-N1 vector DNA was mixed with 100 μl of 150 mM NaCl and then 6.6 μl of ExGen 500, and the DNA mixture was pulse-centrifuged at 3000 rpm for 10 sec. After being incubated for 10 min at room temperature, the DNA mixture was added to each well of a 35 mm culture dish containing fetal fibroblasts grown to 60% confluency, followed by incubation in a CO₂ incubator.

### EXAMPLE 5

### Selection, Proliferation and Cryo-Preservation of Nuclear Donor Cells Transfected with GFP Gene

The pig fetal fibroblasts transfected with GFP genes using three different transfection reagents were cultured for 3-5 days until reaching complete confluency, and detached and separated into single cells by trypsinization. The single cells were observed under a microscope equipped with a UV filter to identify cells expressing GFP protein.

To select only cells expressing GFP protein, the cells was incubated a culture medium supplemented with neomycin for 3 weeks, in which neomycin was added to the medium at a concentration of 400 μg/ml at intervals of 4-5 days. After selection, formed colonies were trypsinized, and cultured in 96-well plates after suitable dilution. The proliferated cells in each well of the 96-well plates were transferred to 24-well plates, and further to 12-well and then 6-well plates, followed by incubation. To investigate whether the GFP gene is integrated into chromosomal DNA of the pig fetal fibroblasts, genomic DNA was isolated from an established clone. Using the isolated genomic DNA, when performing PCR using a primer set designated as the following sequences: 5′-GCGATGCCACCTACGGCAAGCTGA-3′ and 5′-GAGCTGCACGCTGCCGTCCTCGAT-3′, and Southern blotting using a GFP probe, it was found that a GFP gene is integrated into chromosomal DNA of the clone. The identified cloned pig fetal fibroblasts were cryo-preserved by suspending the proliferated cells in a freezing medium prepared using a 10% FCS-containing culture medium and 15% FCS, placing the suspended cells at 4° C. for 2 hrs and then at −70° C. for 12 hrs, and storing the frozen cells at −150° C.

### EXAMPLE 6

### Preparation of Recipient Oocytes

Follicles of about 3-6 mm in diameter were aspirated from pig ovary collected from a slaughterhouse using a 5 ml syringe with an 18-gauge needle. After transferring the follicles to a 100 mm dish having square lattice (1×1 cm) lines, oocytes surrounded by sufficient cumulus cells and having homogeneous cytoplasm were selected. The selected oocytes were washed with 2 ml of NCSU23-W medium in a 35 mm culture dish three times, and finally washed with NCSU23-M medium. Thereafter, FCS-free NCSU23-M medium was supplemented with 10% porcine follicular fluid (PFF), GTH, PMSG, hCG and 10 ng/ml of EGF, and 480 μl of the medium was aliquotted into each well of 4-well plates. 50-60 immature oocytes were put into each well of the plates, and incubated for 22 hrs under 5% CO₂. Then, the oocytes were matured in vitro in NCSU23-M medium not containing the hormones as described above for 20-22 hrs.

### EXAMPLE 7

### Somatic Cell Nuclear Transfer

The recipient oocytes prepared in Example 4 were washed with NCSU23-W medium once, and transferred into NCSU23-W containing 0.1% hyaluronidase. Then, cumulus cells were eliminated from the recipient oocytes. The denuded oocytes were transferred into a cytochalasin B solution prepared by mixing 1 μl of cytochalasin B (Sigma Chemical Co., USA) dissolved in DMSO (dimethyl sulfoxide) at a concentration of 7.5 mg/ml with 1 ml of NCSU23-W medium supplemented with 10% FCS. After fixing the denuded oocytes using a micromanipulator, a holding pipette was rubbed with a sharp micropipette penetrating the zona pellucida of the oocytes to form a slit. Then, 10-15% of cytoplasm was removed from the oocytes by squeezing on their upper part with the sharp micropipette, resulting in production of enucleated oocytes.

The nuclear donor cells prepared in advance were transferred into the enucleated recipient oocytes. First, a 4 μl injection microdroplet was placed on the middle of an upper part of a working dish using a PHA-P (phytohemagglutinin) solution prepared by mixing 100 μl of a PHA-P stock solution prepared by dissolving 5 mg of PHA-P in 10 ml of NCSU23-W medium with 400 μl of NCSU23-W medium. Then, two microdroplets for nuclear donor cells were made above and below the injection microdroplet of the working dish using 4 μl of PBS containing 0.5% FCS. After covering the microdroplets with mineral oil, the working dish was placed on a micromanipulator plate. The enucleated oocytes in NCSU-M medium were washed with NCSU-W medium three times, and transferred into the injection microdroplet. Then, the nuclear donor cells were transferred into the injection microdroplet using an injection pipette. Using the injection pipette, cells identified to express GFP or cells having a GT gene knocked out were injected into the perivitelline space of the enucleated recipient oocytes through the slit (FIG. 3). The resulting transgenic nuclear transfer (NT) embryos were washed with NCSU-W medium three times, and placed into NCSU-W medium.

### EXAMPLE 8

### Cell Fusion and Activation

The transgenic NT embryos were subjected to electrofusion using a BTX Electro cell manipulator (BTX, USA), as follows. 15 μl of a mannitol solution (see Table 4) was added to the NCSU23-W medium containing the NT embryos using a mouth pipette for washing, followed by incubation for 1 min. The NT embryos were incubated for 1 min in a mannitol solution containing NCSU23-W medium, and suspended in the mannitol solution used for their washing, using the mouth pipette. The NT embryos were placed in a chamber with electrodes at each end, containing a mannitol solution and connected to the BTX Electro cell manipulator, in an orientation in which the nuclear donor cells face to the cathode. Thereafter, cell fusion of the NT embryos was induced by applying once a DC pulse of 1.8 kV/cm for 30 μsec. Within 20 min after the electric stimulation, the NT embryos were viewed under a microscope to determine whether cell fusion was achieved, where unfused NT embryos were subjected to electrofusion again. The NT embryos identified to be fused were transferred into NCSU23-W medium, where the NT embryos were activated.

TABLE 4
Mannitol solution

| Components | Conc. | |
|---|---|---|
| Mannitol | 280 | mM |
| HEPES | 0.5 | mM |
| CaCl₂ | 0.1 | mM |
| MgSO₄ | 0.1 | mM |
| BSA | 0.05% | (w/v) |

### EXAMPLE 9

### In Vitro Culturing of Nuclear Transfer Embryos

After being activated in NCSU23-W medium, the electrofused transgenic NT embryos were incubated in NCSU23-D medium. After 4 days of culturing, the NCSU23-D medium was supplemented with 10% FCS. On day 7, each of the transgenic NT embryos was evaluated for development to the blastocyst stage and GFP expression, where GFP expression was investigated under UV illumination (FIG. 4).

### EXAMPLE 10

### Comparison of Development Levels of NT Embryos According to Use of Nuclear Donor Cells Transfected with GFP Gene or not

To evaluate negative or positive effects of introduction of GFP gene into nuclear donor cells on development of nuclear transfer embryos, the nuclear donor cells transfected with a GFP gene, prepared in Example 4, and normal somatic fibroblast cells were subjected to somatic cell nuclear transfer according to the same method in Examples 6 to 9.

In the resulting nuclear transfer embryos, division rates, development rates to the blastocyst stage and cell number in the blastocyst stage were analyzed (see Table 5). As shown in Table 5, it was found that there is no significant difference in development levels of the nuclear transfer embryos between the cases of introducing the GFP gene into the donor cells or not, indicating that the introduction of the GFP gene into fibroblast cells does not affect the development of nuclear transfer embryos.

TABLE 5
Comparison of development levels of nuclear transfer embryos
according to use of nuclear donor cells transfected with a GFP
gene or not

| | Fused | | Development | Cell number |
|---|---|---|---|---|
| | oocyte | | rate to the | in the |
| | cell | Division rate | blastocyst | blastocyst |
| | number | (%) | stage (%) | stage |
| Introduction of | 5031 | 2388(47.5) | 357(15.0) | 44.3 ± 15.1 |
| GFP gene | | | | |
| No introduction | 6681 | 3106(46.5) | 437(14.1) | 46.3 ± 6.4 |
| of GFP gene | | | | |

### EXAMPLE 11

### Comparison of Development Levels of NT Embryos According to Introduction Methods of GFP Gene into Nuclear Donor Cells

To evaluate development levels of nuclear transfer embryos according to transfection reagents used in introduction of GFP gene into nuclear donor cells, nuclear donor cells were transfected with a GFP gene using each of the three transfection reagents used in Example 4, and somatic cell nuclear transfer was performed according to the same method as in Examples 6 to 9.

In the resulting nuclear transfer embryos, division rates, development rates to the blastocyst stage and cell number in the blastocyst stage were analyzed (see Table 6). As shown in Table 6, below, it was found that there is no significant difference in development levels of the nuclear transfer embryos among the three cases, indicating that different methods using different transfection reagents and method do not affect the development levels of the nuclear transfer embryos.

TABLE 6
Comparison of development levels of nuclear transfer embryos
according to introduction methods of GFP gene into nuclear
donor cells

| | Fused | | Development | Cell number |
|---|---|---|---|---|
| | oocyte | | rate to the | in the |
| Transfection | cell | Division rate | blastocyst | blastocyst |
| reagent | number | (%) | stage (%) | stage |
| Untransfected | 6681 | 3106(46.5) | 437 (14.1) | 47.4 ± 13.1 |
| cells | | | | |
| LipofectAmine | 1041 | 502(48.2) | 70 (13.9) | 53.3 ± 11.3 |
| FuGENE 6 | 2967 | 1401(47.2) | 221 (15.7) | 54.4 ± 12.7 |
| ExGen 500 | 1023 | 485(47.4) | 67 (13.8) | 46.3 ± 6.4 |

### EXAMPLE 12

### Transplantation of NT Embryos into Surrogate Mothers

To transfer the nuclear transfer embryos carrying a GFP gene or a knocked out GT gene, prepared in Examples 1 to 11, into surrogate mothers, normal porcine individuals were selected among sows not suffering from maternal diseases and having a regular estrus cycle.

After selecting good quality embryos from the in vitro cultured transgenic nuclear transfer embryos, the selected nuclear transfer embryos were injected 2 cm-deep of the oviduct, close to the ovary (FIG. 5), together with phosphate-buffered saline containing 20% FCS. In detail, the surrogate sows were anesthetized by being intramuscularly injected with the general anesthetic atropine at an amount of 1 mg/kg body weight and then with the tranquilizer azaperrone (Stresnil, P/M; Mallinckrodt) at an amount of 24 mg/kg, and, after 10 min, with ketamine HCl at an amount of 20 mg/kg. Local anesthetization of the region surrounding the skin to be cut was achieved by injection of a 2% lidocaine solution. According to a general laparectomy method, the abdomen of the sows was opened by making a vertical incision about 7 cm long in the middle of the abdomen, while not allowing blood to flow into the inside of the abdomen. The ovary, oviduct and uterus were drawn to the opened region of the abdomen by stimulating the inside of the abdomen by hands. After finding the opened region of the oviduct, carefully handling the ovary, a Tom cat catheter (50 cm, 5 French, open ended catheter, Williams A Cook, MO 63103) equipped with a 1.0 ml tuberculin syringe (Latex free, Becton Dickinson & CO. Franklin lakes, NJ 07417) was inserted 2 cm deep of the oviduct (FIG. 5).

After securing sufficient space at the front of the inserted catheter, the transgenic NT embryos were injected through the catheter. After confirming successful injection of the transgenic NT embryos using a microscope, 500 ml of a physiological saline solution containing antibiotics was injected into the inside of the abdomen. Then, the opened abdomen was sutured with biosorbent suture thread. After the surgery, a broad range of antibiotics was administered to the surrogate sows for 5 days to prevent infection.

### EXAMPLE 13

### Evaluation of Pregnancy of the Surrogate Sows and Production of Live Offspring Expressing GFP and Carrying a Knocked Out GT Gene

4 weeks after the transplantation of the transgenic NT embryos into the surrogate sows, the surrogate mothers were evaluated for pregnancy by an ultrasonic diagnostic system.

Thereafter, the ultrasonic diagnosis was carried out every two weeks to monitor the pregnancy of the surrogate mothers. 114 days after the embryonic transplantation, 7 cloned piglets were born from the surrogate mothers expressing GFP, and 3 cloned piglets were born from the GT gene knock-out surrogate mothers.

### EXAMPLE 14

### Genetic Analysis of Transgenic Cloned Pigs

Genetic analysis of the live offspring produced in Example 13 was carried out by molecular biological methods, and their phenotype was evaluated with the naked eye.

The live offspring were evaluated for GFP expression and introduction of the knocked out GT gene by the naked eye, as well as by performing Southern blotting, Western blotting and cell culture using their tissues.

First, the offspring were evaluated for GFP expression by investigating induction of green color in their skin, mouths and tongues with the naked eye. Also, to investigate GFP expression in the offspring, genomic DNA from the offspring was analyzed by Southern blotting, and protein samples of some tissues were analyzed by Western blotting. As a result, the offspring were found to express GFP. In addition, when analyzing the live offspring born from the surrogate mothers into which the embryos carrying a knocked out GT gene by Southern blotting, the offspring were found to have a knocked out GT gene.

### INDUSTRIAL APPLICABILITY

As described hereinbefore, the present invention provides a cloned pig expressing GFP and a cloned pig carrying a GT gene knocked out by transfecting somatic cells with a GFP gene or a disrupted GT gene, and nuclear transfer of the resulting somatic cells into recipient oocytes, thereby making it possible to produce an animal disease model in a large-scale, as well as an animal able to supply organs transplantable into humans without hyperacute immune rejection.

The present invention has been described in an illustrative manner, and it is to be understood that the terminology used is intended to be in the nature of description rather than of limitation. Many modifications and variations of the present invention are possible in light of the above teachings. Therefore, it is to be understood that within the scope of the appended claims, the invention may be practiced otherwise than as specifically described.

1. A method of producing a cloned pig expressing a green fluorescent protein gene, comprising the steps of:
(a) preparing a nuclear donor cell by culturing a cell line collected from a pig;
(b) mixing pEGFP-N1 and a lipid component or non-lipid cationic polymer vehicle to form lipid (or cationic polymer)-DNA complexes, and adding the resulting complexes to a culture medium of the nuclear donor cell and further culturing the nuclear donor cell to introduce said GFP gene thereinto and express said GFP gene therein;
(c) transferring the transfected nuclear donor cell into an enucleated pig recipient oocyte to generate a transgenic nuclear transfer embryo, and activating said nuclear transfer embryo; and
(d) transplanting the nuclear transfer embryo into a surrogate mother pig to produce live offspring.

2. The method as set forth in claim 1, wherein the lipid component at the step (b) is FuGENE 6 or LipofectAminePlus.

3. The method as set forth in claim 1, wherein the non-lipid cationic polymer is ExGen 500.

4. A porcine nuclear transfer embryo “SNU-P1 [Porcine NT Embryo]”, which is prepared according to the steps (a) to (c) of claim 1, and deposited at KCTC (Korean Collection for Type Cultures) under accesssion number KCTC 10145BP.

5. A cloned pig expressing a green fluorescent protein gene, which is produced from the porcine nuclear transfer embryo “SNU-P1 [Porcine NT Embryo]” of claim 4 by transplanting the nuclear transfer embryo into a surrogate mother pig to produce live offspring.

6. A method of producing a cloned pig having an alpha-1,3-galactosyltransferase gene knocked out, comprising the steps of:
(a) preparing a nuclear donor cell by culturing a somatic cell line collected from a pig;
(b) isolating an alpha-1,3-galactosyltransferase (GT) gene clone from a pig genomic BAC library, and constructing a gene targeting vector using the isolated GT gene, wherein the vector carries a GT gene modified by substituting a portion of a wild-type GT gene with a gene encoding a selectable marker by homologous recombination to suppress expression of a normal GT protein;
(c) mixing the vector with a lipid or non-lipid component to form lipid (or non-lipid)-DNA complexes, and adding the resulting complexes to a culture medium of the nuclear donor cell to allow gene targeting by introducing the recombinant GT gene into the nuclear donor cell;
(d) transferring the nuclear donor cells transfected with the recombinant GT gene into an enucleated pig recipient oocyte to generate a transgenic nuclear transfer embryo, and activating the nuclear transfer embryo; and
(e) transplanting the nuclear transfer embryo into a surrogate mother pig to produce live offspring.

7. The method as set forth in claim 6, wherein the cell line collected from the pig at the step (a) is a fetal fibroblast cell.

8. The method as set forth in claim 6, wherein the gene targeting vector at the step (b) is constructed not to have an exogenous promoter by a promoter trap method.

9. The method as set forth in claim 6, wherein the gene targeting vector at the step (b) comprises a nucleic acid sequence corresponding to a part of intron 8, exon 9 and a part of intron 9 of a GT gene, wherein an AvaI-DraIII fragment of said exon 9 is substituted with a nucleic acid sequence encoding a puromycin-resistant gene linked to a SV 40 poly(A) sequence.

10. The method as set forth in claim 6, wherein the lipid component at the step (c) is FuGENE6.

11. A porcine nuclear transfer embryo “SNU-P2 [Porcine NT Embryo]”, which is prepared according to the steps (a) to (d) of claim 6, and deposited KCTC (Korean Collection for Type Cultures) under accesssion number KCTC 10146BP.

12. A cloned pig having an alpha-1,3-galactosyltransferase gene knocked out, which is produced from the porcine nuclear transfer embryo “SNU-P2 [Porcine NT Embryo]” of claim 11 by transplanting the nuclear transfer embryo into a surrogate mother pig to produce live offspring.

13. A vector carrying a nucleic acid sequence corresponding to a part of intron 8, exon 9 and a part of intron 9 of a GT gene, wherein an AvaI-DraIII fragment of said exon 9 is substituted with a nucleic acid sequence encoding a puromycin-resistant gene linked to a SV 40 poly(A) sequence.
